(19) United States
(12) Patent Application Publication
(10) Pub. No.: US 2019/0070181 A1
(43) Pub. Date: Mar. 7, 2019

(54) FORMULATION OF TICAGRELOR OR PHARMACEUTICALLY ACCEPTABLE SALT THEREOF

(71) Applicant: Jiangsu Hengrui Medicine Co., Ltd., Lianyungang (CN)

(72) Inventors: Hao CHEN, Lianyungang (CN); Xiaochen PAN, Lianyungang (CN); Yun LU, Lianyungang (CN)

(21) Appl. No.: 16/122,992

(22) Filed: Sep. 6, 2018

Related U.S. Application Data
(60) Provisional application No. 62/654,598, filed on Apr. 9, 2018.
(60) Provisional application No. 62/554,619, filed on Sep. 6, 2017.

(51) Int. Cl. A61K 31/519; A61K 9/20

(52) U.S. Cl. CPC A61K 31/519; A61K 9/2031; A61K 9/146; A61K 9/205; A61K 9/2077; A61K 9/2027

(57) ABSTRACT

The present invention relates to a formulation of ticagrelor or a pharmaceutically acceptable salt thereof. Specifically, the present invention relates to an improved formulation of ticagrelor or a pharmaceutically acceptable salt thereof, which is administered once a day. In the present invention, the plasma concentration of ticagrelor in a subject is greater than about 0.2 μg/mL within 2 hours; and the plasma concentration of ticagrelor in a subject is greater than about 0.2 μg/mL at 12 hours after administration; and the maximum plasma concentration (Cₘₐₓ) of ticagrelor or a pharmaceutically acceptable salt thereof in a subject is between about 0.2 μg/mL and about 0.8 μg/mL. The formulation of the present invention can reduce the frequency of administration, thereby improving patient compliance and reducing the risk of myocardial infarction or stroke induced by acute thrombosis which is caused by missed administration of ticagrelor.

### FIELD OF THE INVENTION

The present invention relates to a formulation of ticagrelor or a pharmaceutically acceptable salt thereof, which is administered orally once a day.

### BACKGROUND OF THE INVENTION

Ticagrelor is a platelet aggregation inhibitor, which is a novel cyclopentyl-triazolo-pyrimidines (CPTP) antiplatelet drug administered orally. Its chemical name is (1S,2S,3R,5S)-3-[7-{[(1R,2S)-2-(3,4-difluorophenyl)cyclopropyl]amino}-5-(propylthio)-3H-[1,2,3]-triazolo[4,5-d]pyrimidin-3-yl]-5-(2-hydroxyethoxy)cyclopentane-1,2-diol. Its molecular formula is C₂₃H₂₈F₂N₆O₄S. It has the structural formula as shown below:

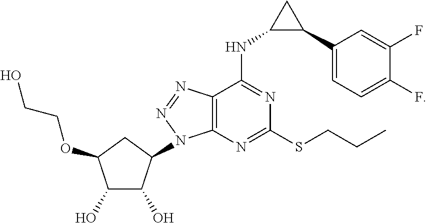 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

This product is useful in patients suffering from acute coronary syndrome (unstable angina, non-ST-elevation myocardial infarction or ST-elevation myocardial infarction), including patients receiving drug therapy and percutaneous coronary intervention (PCI), to reduce the incidence of thrombotic cardiovascular events. Compared to Clopidogrel, this product can reduce the incidence of cardiovascular death, myocardial infarction or stroke composite end point, and the difference between the two treatment groups lies in cardiovascular death and myocardial infarction, whereas there is no difference in stroke.

Ticagrelor is a member of the chemical classification of cyclopentyl-triazolo-pyrimidines (CPTP). CPTP is a selective adenosine diphosphate (ADP) receptor antagonist, which acts on the P2Y12ADP receptor to inhibit the ADP-mediated platelet activation and aggregation, and has a mechanism similar to the thienopyridines (such as Clopidogrel). However, the difference lies in that the interaction between ticagrelor and platelet P2Y12 ADP receptor is reversible, without conformational change and signal transmission, and the platelet function will be recovered rapidly after drug withdrawal.

In ONSET/OFFSET research on patients with stable coronary heart disease receiving aspirin treatment, ticagrelor shows a rapid onset of pharmacological effects. The mean inhibition of platelet aggregation (IPA) reaches 41% at 0.5 hour after administration of 180 mg loading dose of ticagrelor, and the IPA reaches a maximum of 89% at 2-4 hours after administration. This effect can be maintained for 2-8 hours.

The pharmacokinetics of ticagrelor is linear until up to 1260 mg. The exposure dosage of ticagrelor and its active metabolite (AR⋅C124910XX) is substantially proportional to the dosage.

Ticagrelor is rapidly absorbed after oral administration, with a median tₘₐₓ of about 1.5 hours. Since ticagrelor is not a prodrug, it acts on P2Y12 receptor directly without having to be activated by liver metabolism, and can rapidly produce AR-C124910XX which is a main circulating metabolite thereof. The drug itself and its metabolite are both active, therefore it can rapidly and potently inhibit the ADP-mediated platelet aggregation, and its efficacy is not affected by liver CYP 2C19 gene polymorphism. Ticagrelor is mainly eliminated by liver metabolism. After administration of radiolabeled ticagrelor, the measured average recovery of the radiation is about 84% (58% in feces and 26% in urine). The average t_{1/2} of ticagrelor is about 7 hours, and that of the metabolite is about 9 hours.

In order to reduce the frequency of administration, thereby improving patient compliance and reducing the risk of myocardial infarction or stroke induced by acute thrombosis which is caused by missed administration of ticagrelor, it is necessary to prepare ticagrelor into a suitable sustained release component, for example, which can be prepared into a pharmaceutical composition administered only once a day.

There are sustained release components associated with ticagrelor currently. Patent application CN102657629A relates to a sustained release tablet system of ticagrelor and a preparation method thereof. In said patent application, a sustained release component of ticagrelor is prepared by applying hydroxypropyl methylcellulose or hydroxypropyl cellulose as a sustained release material. The present inventor attempted to prepare a sustained release matrix tablet by using cellulose polymer-hydroxypropyl methylcellulose polymer disclosed in patent application CN1102657629A through a conventional tableting method, but found an incomplete release at the later stage. It is supposed that the result is related to the solubility of ticagrelor, since ticagrelor is almost insoluble in water.

By screening a large number of sustained release materials, the present invention demonstrates that a sustained release component of ticagrelor having good sustained release effect can not be prepared through a conventional granulation tableting or direct tableting method by using common sustained release matrix materials such as ethyl cellulose, polymethacrylate, polyvinyl alcohol and the like. However, the inventor unexpectedly find that polyoxyethylene and polyvinyl acetate polymers or alginate are suitable as the sustained release matrix material for ticagrelor and a pharmaceutically acceptable salt thereof, and the sustained release component of ticagrelor or a pharmaceutically acceptable salt thereof can be prepared through a conventional granulation tableting or direct tableting method. Moreover, a better sustained release effect is obtained, compared to the sustained release material of celluloses used in patent application CN1102657629A.

### SUMMARY OF THE INVENTION

The object of the present invention is to provide a sustained release component of ticagrelor or a pharmaceutically acceptable salt thereof, which can moderate the peak-valley phenomenon of plasma concentration so as to improve the drug efficacy and security, as well as reduce the frequency of administration to improve patient compliance.

In an aspect, the present invention provides a formulation administered once a day, comprising ticagrelor or a pharmaceutically acceptable salt thereof, wherein the formulation exhibits the following profile after administration to a subject in need thereof:

(a) the plasma concentration of ticagrelor in the subject is greater than about 0.2 μg/mL, preferably greater than about 0.3 μg/mL within 2 hours; and

(b) the plasma concentration of ticagrelor in the subject is still greater than about 0.2 μg/mL at 12 hours after administration; and

(c) the maximum plasma concentration (Cₘₐₓ) of ticagrelor or a pharmaceutically acceptable salt thereof in the subject is between about 0.2 μg/mL and about 0.8 μg/mL, preferably between 0.4 μg/mL to about 0.6 μg/mL.

In a preferred embodiment, the formulation provided in the present invention comprises a sustained release component, wherein the sustained release component comprises ticagrelor or a pharmaceutically acceptable salt thereof and a sustained release matrix material, wherein the sustained release matrix material is one or more selected from the group consisting of polyoxyethylene, a mixture of polyvinyl acetate and polyvinylpyrrolidone, a single alginate salt, and a mixture of alginates, wherein the alginate is preferably sodium alginate.

Since ticagrelor is almost insoluble in water, the matrix-type sustained release formulation, which is prepared according to a conventional process, always can not achieve a desired effect, the drug tends to dissolve too slowly or incompletely. In an embodiment of the present invention, the particle size of ticagrelor in the sustained release component lies in that D(90) is less than 30 microns, preferably less than 15 microns.

In another embodiment of the present invention, ticagrelor or a salt thereof in the sustained release component is present in the form of a solid dispersion, wherein the solid dispersion comprises a vehicle, the vehicle is one or more selected from the group consisting of povidone, copovidone, polyethylene glycol, poloxamer, and eudragit. The weight ratio of the vehicle material to ticagrelor or a salt thereof can be from 1:0.1 to 1:10, preferably from 1:5 to 5:1, more preferably from 1:2 to 3:1, and most preferably from 1:1 to 2:1. The vehicle material is preferably selected from the group consisting of povidone, copovidone, and a mixture thereof. When the mixture of povidone and copovidone is selected, the weight ratio of the two can be random, for example from 1:0.1 to 1:10, preferably from 1:5 to 5:1, more preferably from 1:1 to 1:3, and most preferably 1:1.

When ticagrelor or a salt thereof in the sustained release component is present in the form of a solid dispersion, the sustained release matrix material is selected from the group consisting of polyoxyethylene, a mixture of polyvinyl acetate and polyvinylpyrrolidone, a single sodium alginate, and a mixture of sodium alginates, preferably polyoxyethylene or a mixture of polyvinyl acetate and polyvinylpyrrolidone (the mixture of polyvinyl acetate and polyvinylpyrrolidone is a commercially available product which has been mixed completely, for example a product marketed under the trade name “Kollidon SR”), and a mixture of the two aforementioned materials (i.e. polyoxyethylene and the mixture of polyvinyl acetate and polyvinylpyrrolidone are present simultaneously), the ratio of the two is not specially defined, preferably, in the mixture, and the weight ratio of polyoxyethylene to the mixture of polyvinyl acetate and polyvinylpyrrolidone is from 1:0.1 to 1:10, preferably from 1:5 to 5:1, more preferably from 1:2 to 3:1, and most preferably 2:1. In another embodiment, the sustained release matrix material is selected from the group consisting of polyoxyethylene, alginate, and a mixture of the two aforementioned materials, preferably, in the mixture, the weight ratio of polyoxyethylene to alginate is from 1:0.1 to 1:10, preferably from 1:5 to 5:1, more preferably from 1:2 to 2:1, and most preferably 3:4.

The molecular weight distribution of polyoxyethylene used in the present invention is relatively wide, from 100,000 Dalton to 7,000,000 Dalton, preferably from 100,000 Dalton to 2,000,000 Dalton, more preferably from 100,000 Dalton to 900,000 Dalton, and most preferably from 100,000 Dalton to 600,000 Dalton. These polyoxyethylenes are commercially available, for example PEO N80, PEO N10, PEO 1105, and PEO N60K.

In a preferred embodiment, the sustained release matrix material of the present invention is polyoxyethylene.

In another preferred embodiment, alginate, preferably sodium alginate, is used as the sustained release matrix material in the sustained release component of the present invention.

In another preferred embodiment, a mixture of sodium alginate and polyoxyethylene is used as the sustained release matrix material in the sustained release component of the present invention, i.e. polyoxyethylene and sodium alginate are used simultaneously. The ratio of the two is not specially defined, preferably, the weight ratio is from 1:0.1 to 1:10, preferably from 1:5 to 5:1, more preferably from 1:2 to 2:1, and most preferably 1:1.

The sustained release component of the present invention comprises 75 mg-250 mg, preferably 180 mg of ticagrelor in each unit. The weight percentage of the matrix materials in the sustained release component can be very broad. Good sustained release component can be formulated when the percentage is greater than 10%. Theoretically, the higher the percentage of the matrix material is, the better the sustained release effect is, but other pharmaceutically factors should be considered during the preparation process. The percentage is preferably from 10% to 95%, more preferably from 10% to 85%, and most preferably from 10% to 50%.

In a preferred sustained release component, the ingredients and contents of the solid dispersion and the sustained release material are shown as follows, wherein the vehicle in the solid dispersion is one or two selected from the group consisting of povidone and copovidone,

| | |
|---|---|
| ticagrelor or a salt thereof | 45-220 mg |
| vehicle | 22.5-440 mg |
| sustained release matrix material | 30-750 mg |

In a further preferred sustained release component, the ingredients and contents of the solid dispersion and the sustained release material are shown as follows, wherein the vehicle in solid dispersion is one or two selected from the group consisting of povidone and copovidone,

| | |
|---|---|
| ticagrelor or a salt thereof | 120-220 mg |
| vehicle | 60-440 mg |
| sustained release matrix material | 75-300 mg |

As a preferred embodiment of the present invention, the sustained release component of the present invention can also comprise an excipient such as a diluent, binder, lubricant and the like. The diluent can be a pharmaceutically acceptable auxiliary material such as lactose, pre-gelatinized starch, microcrystalline cellulose, calcium hydrogen phosphate and the like; the binder can be a pharmaceutically acceptable auxiliary material such as polyvinylpyrrolidone, starch, methylcellulose and the like; the lubricant can be a pharmaceutically acceptable auxiliary material such as magnesium stearate, glyceryl behenate, hydrogenated vegetable oil and the like.

In an embodiment, the sustained release component of the present invention comprises ticagrelor or a pharmaceutically acceptable salt thereof, polyoxyethylene, and magnesium stearate. In a further preferred embodiment, the polyoxyethylene has a molecular weight ranging from 100,000 Dalton to 7,000,000 Dalton, preferably from 100,000 Dalton to 2,000,000 Dalton, more preferably from 100,000 Dalton to 900,000 Dalton, and most preferably from 100,000 Dalton to 600,000 Dalton.

In another embodiment, the sustained release component of the present invention comprises ticagrelor or a pharmaceutically acceptable salt thereof, a mixture of polyvinyl acetate and polyvinylpyrrolidone, and magnesium stearate; or further comprises microcrystalline cellulose in addition to the aforementioned ingredients.

In another embodiment, the sustained release component of the present invention comprises ticagrelor or a pharmaceutically acceptable salt thereof, sodium alginate, and magnesium stearate; or further comprises microcrystalline cellulose in addition to the aforementioned ingredients.

In another embodiment, the sustained release component of the present invention comprises ticagrelor or a pharmaceutically acceptable salt thereof, polyoxyethylene, a mixture of polyvinyl acetate and polyvinylpyrrolidone, and magnesium stearate; or further comprises lactose in addition to the aforementioned ingredients.

When ticagrelor or a salt thereof of the present invention is present in the form of a solid dispersion, the solid dispersion can be prepared by a conventional process, for example solvent method, melting method, solid phase deposit evaporation method, grinding in a ball mill and the like. Preferably, the solvent method is used in the present invention to prepare the solid dispersion, i.e., the drug and the vehicle are dissolved into an appropriate solvent, and the solid dispersion is obtained after removing the organic solvent. In the present invention, the solvent used is selected from the group consisting of ethanol, acetone, ethyl acetate, dichloromethane and the like, preferably ethanol; the vehicle in the solid dispersion is one or more selected from the group consisting of povidone, copovidone, polyethylene glycol, poloxamer, and eudragit, preferably povidone and copovidone.

The sustained release component in the formulation of the present invention can be prepared by a conventional granulation tableting or direct tableting technique, preferably direct tableting technique. The sustained release component with good sustained release effect can be prepared by a simple direct tableting technique, by combining together with some excipients, for example microcrystalline cellulose, pre-gelatinized starch, lactose, mannitol, calcium hydrogen phosphate, glyceryl behenate, magnesium stearate and the like.

The preparation method comprises the following steps of:

1. premixing the crushed ticagrelor or a pharmaceutically acceptable salt thereof with the sustained release matrix material;

2. mixing the premixed mixture of ticagrelor or a pharmaceutically acceptable salt thereof and the sustained release matrix material obtained from Step 1 with the residual excipients;

3. tableting the granule obtained from Step 2 and coating it.

The product has good stability. The preparation process is simple, reproducible and has a high degree of industrialization. Large scale production can be performed with a conventional production equipment.

In addition, the dissolution rate comparison is performed between the ticagrelor composition prepared with hydroxypropyl methylcellulose according to CN1102657629A and the sustained release component of ticagrelor prepared according to the present invention. The result demonstrates that the sustained release component of pharmaceutically acceptable salt of ticagrelor prepared according to the present invention releases more completely at the later stage, and the release behavior is more controllable.

In a preferred embodiment of the present invention, the formulation comprises any of the aforementioned sustained release components, and a rapid release component comprising ticagrelor.

The rapid release component also comprises a pharmaceutically acceptable excipient, wherein the excipient comprises one of diluent, binder, and lubricant, or any combination of them. The diluent can be one or more selected from the group consisting of lactose, microcrystalline cellulose, calcium hydrogen phosphate, and mannitol. The binder can be one or more selected from the group consisting of polyvinylpyrrolidone, starch, carboxymethyl cellulose, and hydroxypropyl methylcellulose. The lubricant can be one or more selected from the group consisting of magnesium stearate, glyceryl behenate, and hydrogenated vegetable oil, preferably magnesium stearate.

In a preferred embodiment of the present invention, in the formulation, the weight ratio of ticagrelor in the sustained release component and the rapid release component is from 1:0.1 to 1:10, preferably from 1:6 to 6:1, more preferably from 1:2 to 6:1, and most preferably from 2:1 to 6:1.

The formulation comprising the sustained release component and the rapid release component can be prepared into the form of a double-layer tablet, or can be prepared into the form of a capsule filled with multiple micro-tablets.

In still another preferred embodiment of the present invention, the formulation comprises a delayed-onset sustained release component, wherein the delayed-onset sustained release component comprises ticagrelor or a salt thereof, and the sustained release component is coated by an enteric material so as to provide a delayed release at pH greater than or equal to 6.0. The enteric material can be selected from the group consisting of acrylic resin, and HPMC-AS; preferably one or more of Eudragit L100, Eudragit S100, and Eudragit L30D-55.

Preferably, ticagrelor in the delayed-onset sustained release component is present in the form of a solid dispersion comprising a vehicle material, wherein the vehicle is preferably one or more selected from the group consisting of povidone, copovidone, polyethylene glycol, poloxamer, and eudragit. The weight ratio of ticagrelor or a salt thereof to the vehicle can be from 1:0.2 to 5, preferably from 1:0.5 to 2, and most preferably 1:1.

Since ticagrelor itself is less soluble, when ticagrelor is prepared into a solid dispersion, and when the amount of the vehicle is low, for example, the weight ratio of the drug to the vehicle is no more than 1:5, preferably no more 1:2, the drug is slowly released and can achieve a sustained release effect, hence the sustained release matrix material does not need to be added.

In another embodiment, the delayed-onset sustained release component can also comprise a sustained release matrix material, preferably selected from the group consisting of polyoxyethylene, a mixture of polyvinyl acetate and polyvinylpyrrolidone, and a mixture of the two aforementioned materials. Preferably, the weight ratio of ticagrelor or a salt thereof to the sustained release matrix material is from 10:1 to 1:1, preferably from 5:1 to 2:1.

The delayed-onset sustained release component can also comprise a pharmaceutically acceptable excipient, wherein the excipient comprises one of diluent, binder, and lubricant, or any combination of them; preferably, the diluent of delayed-onset sustained release component is one or more selected from the group consisting of lactose, microcrystalline cellulose, mannitol, and calcium hydrogen phosphate; preferably, the binder of delayed-onset sustained release component is one or more selected from the group consisting of polyvinylpyrrolidone, starch, carboxymethyl cellulose, and hydroxypropyl methylcellulose; preferably, the lubricant of delayed-onset sustained release component is one or more selected from the group consisting of magnesium stearate, glyceryl behenate, and hydrogenated vegetable oil, and more preferably magnesium stearate.

In a more preferred embodiment, the formulation also comprises an immediate release component of ticagrelor or a salt thereof in addition to the delayed-onset sustained release component, the immediate release component also comprises a pharmaceutically acceptable excipient, wherein the excipient comprises one of diluent, binder, and lubricant, or any combination of them; preferably, the diluent of immediate release component is one or more selected from the group consisting of lactose, microcrystalline cellulose, mannitol, and calcium hydrogen phosphate; preferably, the binder of immediate release component is one or more selected from the group consisting of polyvinylpyrrolidone, starch, carboxymethyl cellulose, and hydroxypropyl methylcellulose; preferably, the lubricant of immediate release component is one or more selected from the group consisting of magnesium stearate, glyceryl behenate, and hydrogenated vegetable oil, and more preferably magnesium stearate.

The weight ratio of ticagrelor or a salt thereof in the delayed-onset sustained release component and the immediate release component can be from 1:0.5 to 1:10, preferably from 1:1 to 1:5, and most preferably 1:2 to 1:4.

In a more preferred embodiment, the formulation also comprises a delayed-onset rapid release component of ticagrelor or a salt thereof in addition to the delayed-onset sustained release component and the immediate release component. In said delayed-onset rapid release component, the rapid release component is coated by an enteric material, and begins to release at pH greater than or equal to 6.0. Preferably, the enteric material is selected from the group consisting of acrylic resin, and HPMC-AS; preferably one or more of Eudragit L100, and Eudragit S100.

The delayed-onset rapid release component also comprises a pharmaceutically acceptable excipient, wherein the excipient comprises one of diluent, binder, and lubricant, or any combination of them; preferably, the diluent of the rapid release component is one or more selected from the group consisting of lactose, microcrystalline cellulose, mannitol, and calcium hydrogen phosphate; preferably, the binder of the rapid release component is one or more selected from the group consisting of polyvinylpyrrolidone, starch, carboxymethyl cellulose, and hydroxypropyl methylcellulose; preferably, the lubricant of the rapid release component is one or more selected from the group consisting of magnesium stearate, glyceryl behenate, and hydrogenated vegetable oil, and more preferably magnesium stearate.

When the formulation comprises three components, the weight ratio of ticagrelor or a salt thereof in the delayed-onset sustained release component, the immediate release component and the delayed-onset rapid release component is shown as follows:

the delayed-onset sustained release component: 1-10 parts;

the delayed-onset rapid release component: 1 part;

the immediate release component: 0.5-5 parts;

preferably, the ratio is shown as follows:

the delayed-onset sustained release component: 2-5 parts;

the delayed-onset rapid release component: 1 part;

the immediate release component: 1-3 parts;

most preferably, the ratio is shown as follows:

the delayed-onset sustained release component: 3 parts;

the delayed-onset rapid release component: 1 part;

the immediate release component: 1-3 parts, for example 2 parts.

The aforementioned delayed-onset sustained release component, immediate release component and delayed-onset rapid release component can be prepared together into the form of a multiple-layer tablet, or can be prepared into the form of a micro-tablet comprising unit dose wherein a different number of said micro-tablets are filled into a capsule depending on the need.

### DESCRIPTION OF THE DRAWINGS

FIG. 1 shows the release profiles of different formulas in Example 1.

FIG. 2 shows the release profiles of different formulas in Example 2.

FIG. 3 shows the release profiles of different formulas in Example 3.

FIG. 4 shows the release profiles of different formulas in Example 4.

FIG. 5 shows the release profiles of different formulas in Example 5.

### DETAILED DESCRIPTION OF THE INVENTION

The present invention will be further illustrated by the following examples without any limitation of the present invention.

The chromatographic condition for determining the dissolution rate: the column packing is octadecyl silane bonded silica gel, and the mobile phase is phosphate buffer (water was added to 10 ml of 1.0 mol/L sodium dihydrogen phosphate solution (the pH of which was adjusted to 3.0 with phosphoric acid) until the volume reached 480 ml, the solution was then shook)-acetonitrile (48:52); the temperature of column is 55° C.; the detection wavelength is 242 nm. The tailing factor of ticagrelor peak should not be greater than 1.5, and the number of theoretical plates is not less than 13,500 calculated by the ticagrelor peak.

### Example 1

Sustained release tablets were prepared by using polyoxyethylene, sodium alginate, or Kollidon SR as a matrix material, respectively. A comparative study was carried out between the above sustained release tablets and the ticagrelor sustained release tablets prepared with hydroxypropyl methylcellulose according to CN1102657629A, and the dissolution rates were evaluated.

| Ingredient | Formula 1 | Formula 2 | Formula 3 | Formula 4 | Formula 5 | Formula 6 | Formula 7 |
|---|---|---|---|---|---|---|---|
| Ticagrelor | 180 mg | 180 mg | 180 mg | 180 mg | 180 mg | 180 mg | 180 mg |
| PEO N80 | 100 mg | — | — | — | — | — | — |
| PEO N10 | — | 50 mg | — | | — | — | — |
| Kollidon SR | — | — | 50 mg | — | — | — | — |
| Sodium alginate | — | — | — | 50 mg | — | — | — |
| HPMC K4M | — | — | — | — | 100 mg | — | — |
| HPMC K100LV | — | — | — | — | — | 100 mg | 150 mg |
| Lactose | 100 mg | 100 mg | 100 mg | 100 mg | 100 mg | 100 mg | 100 mg |
| Microcrystalline | 165 mg | 165 mg | 165 mg | 165 mg | 165 mg | 115 mg | 115 mg |
| cellulose | | | | | | | |
| Magnesium stearate | 5 mg | 5 mg | 5 mg | 5 mg | 5 mg | 5 mg | 5 mg |

Ticagrelor: particle size D90 was about 8 micron.

Ticagrelor, the selected sustained release materials, lactose and microcrystalline cellulose were premixed, and then magnesium stearate was added. The mixture was well mixed, and then compressed into tablets to obtain matrix-type sustained release tablets of ticagrelor.

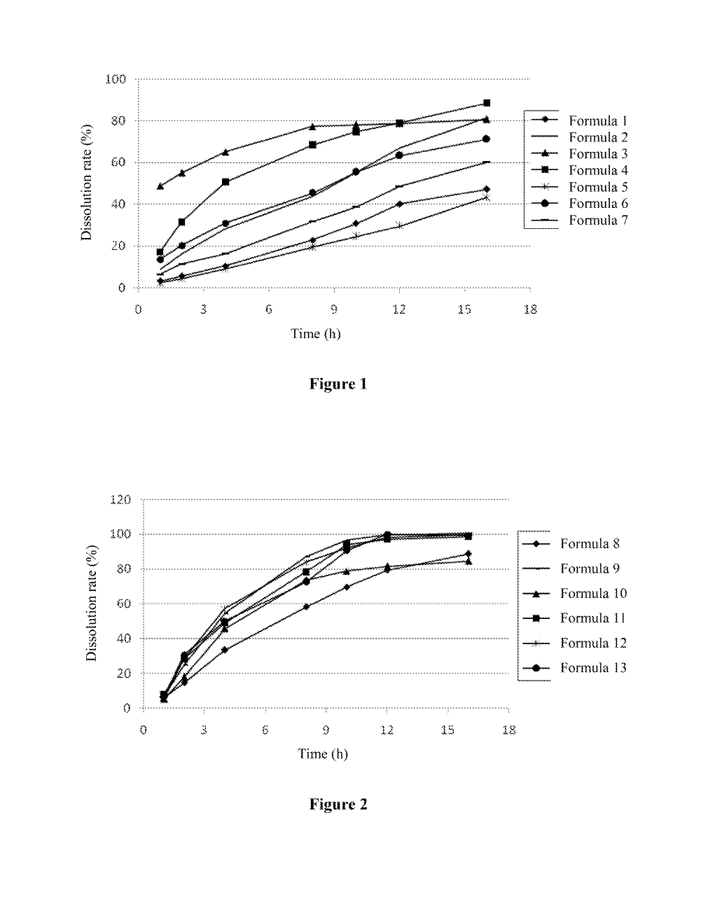

The dissolution rate of sustained release tablets in 900 ml of 0.2% Tween 80 under the condition of 50 rpm was determined by HPLC. The results are shown in Table 1. The release profiles are shown in FIG. 1.

TABLE 1
The results of dissolution rates of different formulas in Example 1

| | Dissolution rate (%) | | | | | | |
|---|---|---|---|---|---|---|---|
| Time (h) | Formula 1 | Formula 2 | Formula 3 | Formula 4 | Formula 5 | Formula 6 | Formula 7 |
| 1 | 3.2 | 8.7 | 48.9 | 17.2 | 2.3 | 13.7 | 6.4 |
| 2 | 5.6 | 16.3 | 55.1 | 31.6 | 4.4 | 20.3 | 11.5 |
| 4 | 10.5 | 28.2 | 65.1 | 50.8 | 9.1 | 31.1 | 16.4 |
| 8 | 15.8 | 43.6 | 77.4 | 68.4 | 19.5 | 45.3 | 31.8 |
| 10 | 30.9 | 55.4 | 78.1 | 74.8 | 24.5 | 55.4 | 38.7 |
| 12 | 40.1 | 67.1 | 78.8 | 79.0 | 29.6 | 63.3 | 48.6 |
| 16 | 47.3 | 81.5 | 80.6 | 88.6 | 43.3 | 71.2 | 60.2 |

Since ticagrelor is almost insoluble in water, all tablets prepared by tableting after directly mixing ticagrelor with different sustained release materials have shown a phenomenon of incomplete dissolution to some extent. It can be seen from Table 1 that when hydroxypropyl methylcellulose was used to prepare a sample according to CN1102657629A, the dissolution rate of the sustained release preparation of ticagrelor prepared with low-viscous hydroxypropyl methylcellulose (HPMCK100LV) is only about 70% at 16 hours. The dissolution rates of ticagrelor compositions prepared with low-viscous polyoxyethylene or sodium alginate according to the present invention can be greater than 80% at 16 hours. When sodium alginate is used as the sustained release material, the dissolution rate can be nearly 90% at 16 hours, and a sustained release for 14 hours to 16 hours can be achieved substantially.

### Example 2

Since ticagrelor is almost insoluble in water, ticagrelor directly mixed with the sustained release material has shown a phenomenon of incomplete dissolution at the later stage. In order to make the in vitro dissolution rate more controllable and meet the dissolution requirement at the later stage, the design idea of “rapid release first, sustained release later” has been provided by the present invention. That is to say, the dissolution rate of the raw material is enhanced by a solid dispersion technique, so as to make the raw material release rapidly; then, the solid dispersion is mixed with the sustained release material and compressed into tablets to obtain sustained release tablets of ticagrelor.

The solid dispersions were prepared by a solvent method using povidone or copovidone as a vehicle, respectively. The resulting solid dispersions were well mixed with polyoxyethylene, microcrystalline cellulose and magnesium stearate, and compressed into tablets to obtain sustained release tablets of ticagrelor.

Formulas:

| | | | Formula | Formula | Formula | Formula |
|---|---|---|---|---|---|---|
| Ingredient | Formula 8 | Formula 9 | 10 | 11 | 12 | 13 |
| Ticagrelor | 180 mg | 180 mg | 180 mg | 180 mg | 180 mg | 180 mg |
| Povidone | 180 mg | 180 mg | 180 mg | 180 mg | — | — |
| Copovidone | — | — | — | | 180 mg | 180 mg |
| PEO N60K | 75 mg | — | — | — | — | — |
| PEO N80 | — | 150 mg | 225 mg | — | 150 mg | — |
| PEO N10 | — | — | — | 225 mg | — | 225 mg |
| Microcrystalline cellulose | 308.5 mg | 233.5 mg | 157.5 mg | 157.5 mg | 233.5 mg | 157.5 mg |
| Magnesium stearate | 7.5 mg | 7.5 mg | 7.5 mg | 7.5 mg | 7.5 mg | 7.5 mg |

The dissolution rate of sustained release tablets in 900 ml of 0.2% Tween 80 under the condition of 50 rpm was determined by HPLC. The results are shown in Table 2. The release profiles are shown in FIG. 2.

TABLE 2
The results of dissolution rates of different formulas in Example 2

| | Dissolution rate (%) | | | | | |
|---|---|---|---|---|---|---|
| Time (h) | Formula 8 | Formula 9 | Formula 10 | Formula 11 | Formula 12 | Formula 13 |
| 1 | 6.5 | 7.6 | 5.3 | 8.1 | 6.0 | 6.6 |
| 2 | 14.7 | 24.9 | 18.1 | 28.5 | 28.7 | 30.9 |
| 4 | 33.6 | 54.7 | 45.8 | 49.2 | 57.3 | 50.1 |
| 8 | 58.3 | 87.3 | 73.8 | 78.4 | 84.1 | 72.5 |
| 10 | 69.8 | 96.7 | 78.8 | 94.1 | 92.3 | 90.8 |
| 12 | 79.4 | 99.9 | 81.7 | 97.2 | 98.1 | 100.1 |
| 16 | 88.9 | 100.4 | 84.7 | 98.7 | 99.7 | 99.3 |

It can be seen from the dissolution rate data of different formulas in Example 2 that when the raw material was solubilized by a solid dispersion technique and then mixed with the auxiliary materials, the dissolution rate of the preparation can be well regulated by adjusting the viscosity and amount of polyoxyethylene. It allows the preparation to completely release within 16 hours, and allows the dissolution rate to be more controllable.

### Example 3

The solid dispersion of ticagrelor was prepared into sustained release preparations by using sodium alginate, Kollidon SR, or polyoxyethylene as the matrix material, respectively. The release behaviors of the preparations were evaluated.

| | Formula | Formula | Formula | Formula | Formula | Formula | Formula | Formula |
|---|---|---|---|---|---|---|---|---|
| Ingredient | 14 | 15 | 16 | 17 | 18 | 19 | 20 | 21 |
| Ticagrelor | 180 mg | 180 mg | 180 mg | 180 mg | 180 mg | 180 mg | 180 mg | 180 mg |
| Copovidone | 180 mg | 180 mg | 180 mg | 180 mg | 180 mg | 180 mg | 180 mg | 180 mg |
| PEO N80 | — | — | | 75 mg | 75 mg | 150 mg | 112.5 mg | — |
| PEO 205 | — | — | — | — | — | — | — | 100 mg |
| PEO N60K | — | — | — | — | — | — | — | 50 mg |
| Kollidon SR | 75 mg | 150 mg | — | 75 mg | 112.5 mg | 112.5 mg | — | 112.5 mg |
| Sodium alginate | — | — | 150 mg | | | — | 150 mg | — |
| Lactose | 75 mg | — | — | — | — | — | — | — |
| Microcrystalline | 233.5 mg | 233.5 mg | 233.5 mg | 233.5 mg | 196.0 mg | 121 mg | 121 mg | 121 mg |
| cellulose | | | | | | | | |
| Magnesium | 7.5 mg | 7.5 mg | 7.5 mg | 7.5 mg | 7.5 mg | 7.5 mg | 7.5 mg | 7.5 mg |
| stearate | | | | | | | | |

Preparation Method:

The vehicle materials and the active pharmaceutical ingredient were prepared into a solid dispersion, which was then well mixed with auxiliaries and compressed into tablets directly to obtain sustained release tablets.

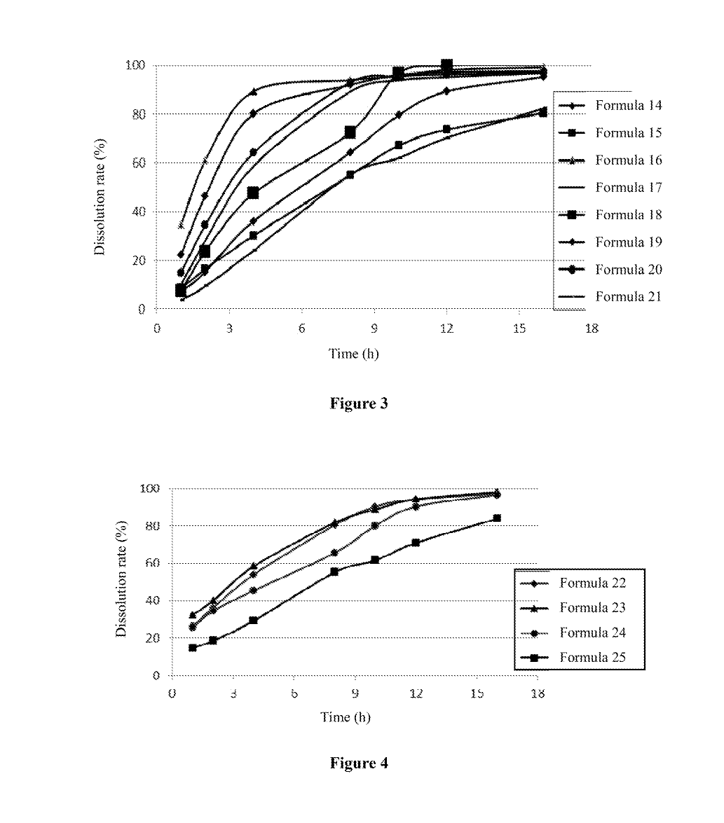

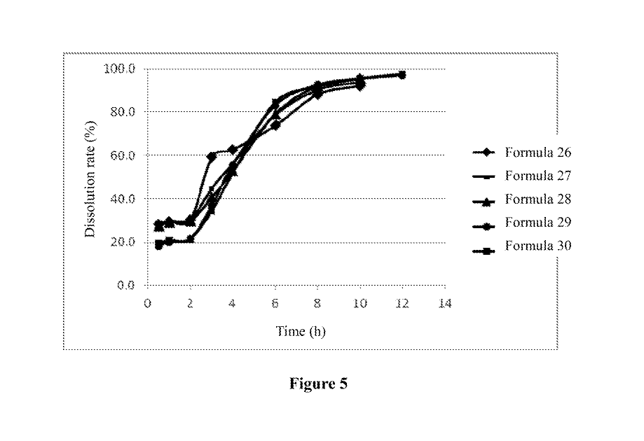

The dissolution rate of sustained release tablets in 900 ml of 0.2% Tween aqueous solution was determined by HPLC. The results are shown in Table 3. The release profiles are shown in FIG. 3.

TABLE 3
The results of dissolution rates of different formulas in Example 3

| | Dissolution rate (%) | | | | | | | |
|---|---|---|---|---|---|---|---|---|
| | Formula | Formula | Formula | Formula | Formula | Formula | Formula | Formula |
| Time (h) | 14 | 15 | 16 | 17 | 18 | 19 | 20 | 21 |
| 1 | 22.4 | 8.7 | 34.7 | 10.2 | 7.6 | 7.1 | 15.0 | 3.8 |
| 2 | 46.5 | 16.5 | 61.1 | 28.2 | 23.7 | 15.4 | 34.5 | 9.5 |
| 4 | 80.3 | 30.2 | 89.5 | 58.8 | 47.5 | 36.2 | 64.2 | 24.2 |
| 8 | 92.1 | 55.1 | 94.1 | 89.3 | 72.6 | 64.3 | 93.1 | 55.2 |
| 10 | 96.2 | 67.1 | 95.7 | 94.0 | 96.7 | 79.6 | 95.6 | 62.0 |
| 12 | 97.2 | 73.7 | 98.1 | 95.3 | 99.9 | 89.4 | 96.4 | 70.3 |
| 16 | 97.7 | 80.5 | 99.2 | 97.0 | 100.2 | 95.4 | 97.1 | 82.3 |

It can be seen from Table 3 and FIG. 3 that sodium alginate and Kollidon SR both have sustained release effects to some extent when being used as the sustained release matrix material. The addition of sodium alginate or Kollidon SR to polyoxyethylene can better control the dissolution rate. A sustained release component with good sustained release effect can be prepared by a direct tableting technique.

### Example 4

The preparation of double-layer tablets: The sustained release component was prepared by a combination of polyoxyethylene, polyvinyl acetate and polyvinylpyrrolidone (PVP) as the matrix material. Ticagrelor, crospovidone, lactose, microcrystalline cellulose, and magnesium stearate were mixed as the rapid release component.

Formulas:

| | | | | Formula |
|---|---|---|---|---|
| | Formula 22 | Formula 23 | Formula 24 | 25 |
| Sustained release | | | | |
| component | | | | |
| Ticagrelor | 126.0 | 126.0 | 126.0 | 153.0 |
| Copovidone | 126.0 | 126.0 | 126.0 | 153.0 |
| PEO N80 | 105.0 | — | — | — |
| PEO N10 | — | 140.0 | — | — |
| PEO 1105 | — | — | 157.5 | — |
| PEO N60K | — | — | — | 127.5 |
| Kollidon SR | 78.8 | 78.8 | 78.8 | 95.6 |
| Microcrystalline | 84.7 | 49.7 | 34.1 | 102.9 |
| cellulose | | | | |
| Magnesium stearate | 5.3 | 5.3 | 5.3 | 6.4 |
| Rapid release | | | | |
| component | | | | |
| Ticagrelor | 54.0 | 54.0 | 54.0 | 27.0 |
| Copovidone | 54.0 | 54.0 | 54.0 | 27.0 |
| Crospovidone | 106.8 | 106.8 | 106.8 | 108.0 |
| Lactose | 70.8 | 70.8 | 70.8 | 129.6 |
| Microcrystalline | 70.8 | 70.8 | 70.8 | 64.8 |
| cellulose | | | | |
| Magnesium stearate | 3.6 | 3.6 | 3.6 | 3.6 |

Preparation method: For Formulas 22, 23, 24 and 25, the sustained release component and the rapid release component were well mixed respectively according to the formula ratio. Double-layer tablets were prepared by a direct tableting technique. The dissolution rate of sustained release tablets in 900 ml of 0.2% Tween aqueous solution was determined by HPLC. The results are shown in Table 4. The release profiles are shown in FIG. 4.

TABLE 4
The results of dissolution rates of different formulas in Example 4

| | Dissolution rate (%) | | | |
|---|---|---|---|---|
| Time (h) | Formula 22 | Formula 23 | Formula 24 | Formula 25 |
| 1 | 26.5 | 32.5 | 25.8 | 14.9 |
| 2 | 36.2 | 40.1 | 34.5 | 18.7 |
| 4 | 54.0 | 58.4 | 45.5 | 29.3 |
| 8 | 80.4 | 81.7 | 65.6 | 55.2 |
| 10 | 90.3 | 88.8 | 80.1 | 61.6 |
| 12 | 94.1 | 94.2 | 90.3 | 70.9 |
| 16 | 97.1 | 97.9 | 96.5 | 83.8 |

It can be seen from Table 4 and FIG. 4 that using a combination of the rapid release component and the sustained release component as double-layer matrix materials can achieve the effect of rapid release at earlier stage and sustained release at later stage.

### Example 5

A composition having an immediate release micro-tablet of ticagrelor and a delayed release micro-tablet of ticagrelor was prepared according to Formulas 25, 26 and 27. The composition comprises a double pulse consisting of a composition of a rapid release micro-tablet and an enteric sustained release micro-tablet; and also comprises a triple pulse consisting of a composition of a rapid release micro-tablet, an enteric rapid release micro-tablet and an enteric sustained release micro-tablet. The enteric rapid release micro-tablet was prepared by coating the aforementioned rapid release micro-tablet with an enteric material. The enteric sustained release micro-tablet was prepared as follows: ticagrelor was dispersed in copovidone as a vehicle material to obtain a solid dispersion, which was then mixed with other auxiliary materials and coated with an enteric material. A sustained release matrix material such as PEO N10 can be added. However, since ticagrelor itself is less soluble, when the amount of vehicle material is low, a sustained release effect can already be achieved, hence the sustained release matrix material does not need to be added.

Formulas:

| | | | | |
|---|---|---|---|---|
| Formula 26 | Formula 26 | Formula 28 | Formula 29 | Formula 30 |

Rapid release micro-tablet

| Ticagrelor | 30 | mg | 30 | mg | 30 | mg | 27 | mg | 40 | mg |
|---|---|---|---|---|---|---|---|---|---|---|
| Mannitol | 34.8 | mg | 34.8 | mg | 34.8 | mg | 37.8 | mg | 56 | mg |
| Calcium hydrogen | 18.9 | mg | 18.9 | mg | 18.9 | mg | 18.9 | mg | 28 | mg |
| phosphate | | | | | | | | | | |
| Sodium carboxymethyl | 2.7 | mg | 2.7 | mg | 2.7 | mg | 2.7 | mg | 4 | mg |
| starch | | | | | | | | | | |
| Hydroxypropyl | — | | — | | — | | 2.7 | mg | 4 | mg |
| cellulose SSL | | | | | | | | | | |
| Hydroxypropylcellulose | 2.7 | mg | 2.7 | mg | 2.7 | mg | — | | — | |
| E5 | | | | | | | | | | |
| Magnesium stearate | 0.9 | mg | 0.9 | mg | 0.9 | mg | 0.9 | g | 1.33 | mg |
| Opadry 85G620008 | — | | — | | — | | 2.7 | mg | 4 | mg |
| Total weight of tablets | 90 | mg | 90 | mg | 90 | mg | 92.7 | mg | 137.33 | mg |
| Number of tablets | 2 | tablets | 2 | tablets | 2 | tablets | 1 | tablet | 1 | tablet |

Enteric rapid release micro-tablet

| Rapid release | 90 | mg | 90 | mg | — | — | — |
|---|---|---|---|---|---|---|---|
| micro-tablet | | | | | | | |
| Eudragit L100 | 10 | mg | 2.5 | mg | — | — | — |
| Eudragit S100 | — | | 7.5 | mg | — | — | — |
| Total weight of tablets | 100 | mg | 100 | mg | — | — | — |
| Number of tablets | 2 | tablets | 1 | tablet | — | — | — |

Enteric sustained release micro-tablet

| Ticagrelor | 30 | mg | 30 | mg | 30 | mg | 31 | mg | 35 | mg |
|---|---|---|---|---|---|---|---|---|---|---|
| Copovidone | 30 | mg | 30 | mg | 30 | mg | 31 | mg | 35 | mg |
| Butylated | — | | — | | — | | 0.0186 | mg | 0.021 | mg |
| hydroxytoluene | | | | | | | | | | |
| Mannitol | — | | — | | — | | 10.4 | mg | 11.7 | mg |
| Microcrystalline | 39 | mg | 29 | mg | 19 | mg | 20.7 | mg | 23.3 | mg |
| cellulose | | | | | | | | | | |
| PEO N10 | — | | — | | 10 | mg | — | | — | |
| Crospovidone | — | | 10 | mg | 10 | mg | — | | — | |
| Croscarmellose Sodium | — | | — | | — | | 10.6 | mg | 12 | mg |
| Colloidal Silicon | — | | — | | — | | 1.1 | mg | 1.2 | mg |
| Dioxide | | | | | | | | | | |
| Magnesium stearate | 1 | mg | 1 | mg | 1 | mg | 1.6 | mg | 1.8 | mg |
| Eudragit L100 | 10 | mg | 2.5 | mg | 2.5 | mg | — | | — | |
| Eudragit S100 | — | | 7.5 | mg | 7.5 | mg | — | | — | |
| Eudragit L30D-55 | — | | — | | — | | 22.2 | mg | 24.9 | mg |
| aqueous | | | | | | | | | | |
| dispersion (solid content | | | | | | | | | | |
| 30%) | | | | | | | | | | |
| Talc powder | — | | — | | — | | 3.3 | mg | 3.8 | mg |
| Triethyl citrate | — | | — | | — | | 0.7 | mg | 0.8 | mg |
| Total weight of tablets | 110 | mg | 110 | mg | 110 | mg | 117 | mg | 132 | mg |
| Number of tablets | 2 | tablets | 3 | tablets | 4 | tablets | 3 | tablets | 4 | tablets |

Preparation method: the rapid release micro-tablets and the sustained release micro-tablets were prepared respectively by a tableting technique, then coated by an enteric material according to the corresponding formula ratio and filled into capsules. The dissolution rates of sustained release capsules (Formulas 26, 27 and 28) in 900 ml of 0.2% Tween hydrochloric acid solution pH 1.0 and 0.2% Tween phosphate buffer pH 6.8 were determined by HPLC. The dissolution rates of sustained release capsules (Formulas 29 and 30) were determined by Ultraviolet and Visible Spectrophotometry. Dissolving medium was 750 mL of 0.2% Tween 80 hydrochloric acid solution pH 1.0 and added 250 mL of 0.2% Tween 80 sodium phosphate solution 0.22 mol/L. The results are shown in Table 5 and Table 6.

TABLE 5
The results of dissolution rates of capsules in Example 5 in different media

| | | Dissolution rate (%) | | |
|---|---|---|---|---|
| Medium | Time (h) | Formula 26 | Formula 27 | Formula 28 |
| 0.2% Tween | 0.5 h | 28.2 | 27.9 | 27.6 |
| pH 1.0 | 1 h | 29.5 | 29.8 | 29.2 |
| | 2 h | 30.3 | 30.5 | 29.9 |
| 0.2% Tween | 3 h | 59.2 | 44.8 | 40.7 |
| pH 6.8 | 4 h | 62.7 | 56.4 | 52.8 |
| | 6 h | 73.8 | 78.7 | 79.2 |
| | 8 h | 88.2 | 90.4 | 92.1 |
| | 10 h | 92.2 | 94.1 | 95.6 |

TABLE 6
The results of dissolution rates of capsules in Example 5 in different media

Dissolution rate (%)

| Medium | Time (h) | Formula 29 | Formula 30 |
|---|---|---|---|
| 0.2% Tween | 0.5 h | 18.2 | 19.8 |
| pH 1.0 | 1 h | 20.2 | 20.9 |
| | 2 h | 21.7 | 21.6 |
| 0.2% Tween | 3 h | 36.8 | 34.4 |
| pH 6.8 | 4 h | 55.7 | 51.9 |
| | 6 h | 83.1 | 84.5 |
| | 8 h | 92.7 | 91.6 |
| | 10 h | 95.5 | 95.5 |
| | 12 h | 97.0 | 97.6 |

### Example 6

Pharmacokinetics (plasma concentration) test and pharmacodynamics (platelet inhibition rate) test in human were carried out selectively with the formulas disclosed in the above examples. The relationship between in vitro release and pharmacokinetics (PK) and pharmacodynamics (PD) was analyzed, so as to explore the in vitro and in vivo correlation of ticagrelor.

In the present invention, randomized, cross, single center, controlled test was carried out in healthy voluntary subjects with different formulas. The pharmacokinetics and pharmacodynamics of ticagrelor in healthy subjects were evaluated in the test. In the test, 180 mg of the formulas of the present invention were administered once a day, and 90 mg of a commercially available rapid release preparation was administered twice a day. Plasma samples were collected to determine the plasma concentration of ticagrelor and platelet inhibition. The samples were analyzed by HPLC/MS/MS.

TABLE 7
Pharmacokinetic parameters of ticagrelor with multiple in vitro release profiles

| | | Main pharmacokinetic parameters | | | | | | |
|---|---|---|---|---|---|---|---|---|
| | Administration | AUC₀₋ₜ | AUC_{0-∞} | Cₘₐₓ | C_{2 h} | C_{12 h} | Tₘₐₓ | T_{1/2} |
| Preparation | protocol | ng/mL * h | ng/mL * h | ng/mL | ng/mL | ng/mL | h | h |
| Formula 19 | 180 mg, QD | 7725 | 7945 | 802 | 530 | 249 | 3.6 | 9.6 |
| Formula 21 | 180 mg, QD | 6283 | 6485 | 593 | 263 | 217 | 3.8 | 9.0 |
| Formula 22 | 180 mg, QD | 7617 | 7799 | 889 | 655 | 213 | 2.9 | 9.2 |
| Formula 24 | 180 mg, QD | 7925 | 8212 | 738 | 423 | 238 | 4.1 | 10.2 |
| Formula 25 | 180 mg, QD | 6344 | 6862 | 565 | 406 | 173 | 3.6 | 11.1 |
| Formula 27 | 180 mg, QD | 8043 | 8257 | 652 | 384 | 308 | 5.8 | 11.5 |
| Formula 30 | 180 mg, QD | 8749 | 8984 | 814 | 362 | 251 | 5 | 9.1 |
| Commercially | 90 mg, BID | 8225 | 8541 | 615 | 497 | 88.1 | 1.6 | 8.7 |
| available | | | | | | | | |
| formulation 1 | | | | | | | | |
| Commercially | 90 mg, BID | 7350 | 7653 | 559 | 447 | 85.2 | 1.5 | 7.8 |
| available | | | | | | | | |
| formulation 2 | | | | | | | | |

Anti-platelet aggregation effect in healthy voluntary subjects: Platelet aggregation inhibition rates were determined before administration and at 2, 4, 8, 12 and 24 hours after administration. The ratios of the platelet inhibition rate of the formulas of the present invention to that of commercially available rapid release preparation are shown in Table 8.

TABLE 8
Pharmacokinetic parameters of ticagrelor with multiple release profiles

| Preparation | 2 h | 4 h | 8 h | 12 h | 24 h |
|---|---|---|---|---|---|
| Formula 19 | 0.97 | 0.95 | 1.23 | 1.01 | 0.97 |
| Formula 21 | 0.91 | 0.89 | 0.96 | 1.15 | 0.74 |
| Formula 22 | 1.08 | 1.08 | 1.25 | 1.01 | 0.94 |
| Formula 24 | 1.05 | 0.99 | 1.17 | 1.09 | 1.04 |
| Formula 25 | 1.14 | 0.99 | 1.16 | 1.24 | 0.66 |
| Formula 27 | 1.09 | 1.04 | 1.11 | 1.10 | 1.06 |
| Commercially available formulation 1 | — | — | — | — | — |

It is known from the data analysis of in vivo pharmacokinetics and in vitro release that the formulation releases a certain amount of drug at the earlier stage of in vitro dissolution, which is helpful in keeping a certain plasma concentration (C₂ₕ), further ensuring the efficacy at 2 and 4 hours, thereby ensuring the rapid onset of drug. The plasma concentration is kept at a certain level after 12 hours, for example at least 0.2 μg/mL, to maintain the efficacy for 24 hours. The maximum plasma concentration (Cₘₐₓ) of the drug of the present invention is equal to or slightly higher than that of the commercially available rapid release preparation, in order to avoid the adverse effect caused by the excess of efficacy at 8 h and 12 h.

Specifically, the plasma concentration of ticagrelor in the subject is greater than about 0.2 μg/mL within 2 hours; and the plasma concentration of ticagrelor in the subject is still greater than about 0.2 μg/mL at 12 hours after administration; and the maximum plasma concentration (Cₘₐ) of ticagrelor or a pharmaceutically acceptable salt thereof in the subject is between about 0.2 μg/mL and about 0.8 μg/mL.

As the present invention has been described in terms of specific embodiments thereof, certain modifications and equivalent variations will be apparent to those skilled in the art and are intended to be included within the scope of the present invention.

1. A formulation administered once a day, comprising ticagrelor or a pharmaceutically acceptable salt thereof, wherein the formulation exhibits the following profile after administration to a subject in need thereof:
a) the plasma concentration of ticagrelor or the salt thereof in the subject is greater than about 0.2 μg/mL within 2 hours; and
b) the plasma concentration of ticagrelor or the salt thereof in the subject is greater than about 0.2 μg/mL at 12 hours after administration; and
c) the maximum plasma concentration (Cₘₐₓ) of ticagrelor or the salt thereof in the subject is about 0.2 μg/mL to about 0.8 μg/mL.

2. The formulation according to claim 1, comprising a sustained release component, wherein the sustained release component comprises ticagrelor or the salt thereof and a sustained release matrix material, wherein the sustained release matrix material is at least one selected from the group consisting of polyoxyethylene, a mixture of polyvinyl acetate and polyvinylpyrrolidone, a single alginate salt, and a mixture of alginate salts.

3. The formulation according to claim 2, wherein the ticagrelor or the salt thereof in the sustained release component has a particle size D(90) is of less than 30 microns.

4. The formulation according to claim 2, wherein ticagrelor or the salt thereof in the sustained release component is present in the form of a solid dispersion, wherein the solid dispersion comprises a vehicle, wherein the vehicle is at least one selected from the group consisting of povidone, copovidone, polyethylene glycol, poloxamer, and eudragit.

5. The formulation according to claim 4, wherein a weight ratio of the vehicle to ticagrelor or the salt thereof in the sustained release component is 1:0.1 to 1:10.

6. The formulation according to claim 5, wherein the sustained release matrix material in the sustained release component is selected from the group consisting of polyoxyethylene, and a mixture of polyvinyl acetate and polyvinylpyrrolidone, or a mixture thereof wherein the mixture has a weight ratio of polyoxyethylene to the mixture of polyvinyl acetate and polyvinylpyrrolidone of 1:0.1 to 1:10.

7. The formulation according to claim 5, wherein the sustained release matrix material in the sustained release component is selected from the group consisting of polyoxyethylene, alginate, and a mixture of polyoxyethylene and alginate, wherein the mixture has a weight ratio of polyoxyethylene to alginate of 1:0.1 to 1:10.

8. The formulation according to claim 2, wherein the polyoxyethylene has a molecular weight ranging from 100,000 Dalton to 7,000,000 Dalton.

9. The formulation according to claim 2, wherein a weight percentage of the sustained matrix material in the sustained release component is 5% to 95%.

10. The formulation according to claim 1, wherein a content of ticagrelor or the salt thereof calculated by ticagrelor is 45 mg to 220 mg.

11. The formulation according to claim 4, wherein the solid dispersion and the sustained release matrix material in the sustained release component have ingredients and contents shown as follows:
ticagrelor or the salt thereof: 45-220 mg;
vehicle: 22.5-440 mg;
sustained release matrix material: 30-750 mg;
wherein the vehicle in the solid dispersion is at least one selected from the group consisting of povidone and copovidone.

12. The formulation according to claim 2, wherein the sustained release component comprises a pharmaceutically acceptable excipient, wherein the excipient comprises one of a diluent, binder, and lubricant, or any combination thereof.

13. The formulation according to claim 2, further comprising a rapid release component of ticagrelor or the salt thereof.

14. The formulation according to claim 13, wherein the rapid release component further comprises a pharmaceutically acceptable excipient, wherein the excipient comprises one of a diluent, binder, and lubricant, or any combination thereof.

15. The formulation according to claim 13, wherein the formulation has a weight ratio of ticagrelor or the salt thereof in the sustained release component to ticagrelor or the salt thereof in the rapid release component of 1:0.1 to 1:10.

16. The formulation according to claim 1, comprising a delayed-onset sustained release component, wherein the delayed-onset sustained release component comprises ticagrelor or the salt thereof, and the delayed-onset sustained release component is coated by an enteric material so as to provide a delayed release at a pH greater than or equal to 6.0.

17. The formulation according to claim 16, wherein the enteric material is selected from the group consisting of acrylic resin, and HPMC-AS.

18. The formulation according to claim 16, wherein ticagrelor or the salt thereof in the delayed-onset sustained release component is present in the form of a solid dispersion, wherein the solid dispersion comprises a vehicle material, wherein the vehicle is selected from the group consisting of povidone, copovidone, polyethylene glycol, poloxamer, eudragit, HPMC-AS and a mixture thereof.

19. The formulation according to claim 18, wherein the formulation has a weight ratio of ticagrelor or the salt thereof to the vehicle of 1:0.2 to 5.

20. The formulation according to claim 18, wherein the delayed-onset sustained release component further comprises a sustained release matrix material.

21. The formulation according to claim 20, wherein the formulation has a weight ratio of ticagrelor or the salt thereof to the sustained release matrix material of 10:1 to 1:1.

22. The formulation according to claim 16, wherein the delayed-onset sustained release component further comprises a pharmaceutically acceptable excipient, wherein the excipient comprises one of a diluent, binder, and lubricant, or any combination of them.

23. The formulation according to claim 16, further comprising an immediate release component of ticagrelor or the salt thereof, wherein the immediate release component comprises a pharmaceutically acceptable excipient, wherein the excipient comprises one of a diluent, binder, and lubricant, or any combination thereof.

24. The formulation according to claim 23, wherein the formulation has a weight ratio of ticagrelor or the salt thereof in the delayed-onset sustained release component to the ticagrelor or the salt thereof in the immediate release component of 1:0.5 to 1:10.

25. The formulation according to claim 23, further comprising a delayed-onset rapid release component of ticagrelor or the salt thereof, wherein the rapid release component is coated by an enteric material, and begins to release at a pH greater than or equal to 6.0.

26. The formulation according to claim 25, wherein the delayed-onset rapid release component further comprises a pharmaceutically acceptable excipient, wherein the excipient comprises one of a diluent, binder, and lubricant, or any combination thereof.

27. The formulation according to claim 25, wherein the formulation has a weight ratio of ticagrelor or the salt thereof in the delayed-onset sustained release component, the immediate release component and the delayed-onset rapid release component shown as follows:
the delayed-onset sustained release component: 1-10 parts;
the delayed-onset rapid release component: 1 part;
the immediate release component: 0.5-5 parts.
